(19) United States
(12) Patent Application Publication
(10) Pub. No.: US 2024/0384255 A1
(43) Pub. Date: Nov. 21, 2024

(54) ENGINEERED MICROMAGMATICS FOR DEPLOYING MICROORGANISMS

(71) Applicant: BATTELLE SAVANNAH RIVER ALLIANCE, LLC, AIKEN, SC (US)

(72) Inventors: ROBIN L. BRIGMON, NORTH AUGUSTA, SC (US); ALEX J. KUGLER, AIKEN, SC (US); CORY L. TRIVELPIECE, AIKEN, SC (US)

(21) Appl. No.: 18/317,314

(22) Filed: May 15, 2023

(51) Int. Cl. C12N 11/14; C12N 1/20

(52) U.S. Cl. CPC C12N 11/14; C12N 1/20

(57) ABSTRACT

A system for deploying a microorganism to a target environment. The system comprises a plurality of micromagmatic particles. Each micromagmatic particle comprises a lyophilized microorganism supported on a glass microparticle having a median particle size from about 1 μm to about 1000 μm as determined by laser diffraction according to ISO 13320:2020.

This invention was made with Government support under Contract No. 89303321CEM00080, awarded by the U.S. Department of Energy. The Government has certain rights in the invention.

### BACKGROUND

Microbial amendments are beneficially used in a plurality of applications, including oil degradation, mining, bioremediation of contaminated waters and soils, agriculture, wastewater treatment, and even within the human body. Cell cultures are a common part of bioremediation strategies, but they can be difficult to deploy. Aerobic bacteria in particular can be extremely difficult to deploy in the field. Cultures can die in transport or fail to proliferate and must be chosen specifically for each deployment environment. Liquid bioremediation cultures are commonly developed and, while they can be deployed in the field, the process is difficult, expensive, and requires considerable regulatory oversight, making it desirable to find alternatives means of deployment.

Bioremediation strategies have also looked to the development and use of biofilms. Biofilms are used in water filtration systems, wastewater remediation, biocatalysts, and the generation of biofuels. Such films often have high cell densities, e.g., 10⁸-10¹¹ cells per gram of wet biomass. While biofilms can form from a single species, they often consist of many different species living together. This diversity aids in their survival and enhances their biotechnological use by allowing coordination of life cycles by staggering the expression of certain genes or proteins. Biofilms also have enhanced gene exchange due to cell/cell proximity and high cell density which increases the resiliency of the bacteria compared to their free-living counterparts. Considerable research has focused on biofilm growth on various surfaces such as rocks, well screens, and ships. Unfortunately, however, these surfaces are of limited biotechnical application and many desirable microbes have been difficult or impossible to develop in the form of biofilms on such surfaces.

Needed in the art are methods and systems for deployment of microorganisms. Methods and systems that can be utilized to store and deploy beneficial microorganisms efficiently and with relative ease, for instance, in the form of a solid supported biofilm, would be of great benefit to the art.

### SUMMARY

In one aspect, a system for deploying a microorganism to a target environment is disclosed. The system comprises a plurality of micromagmatic particles. Each micromagmatic particle comprises a lyophilized microorganism supported on a glass microparticle having a median particle size from about 1 μm to about 1000 μm, as determined by laser diffraction according to ISO 13320:2020.

Also disclosed is a method for treating a target environment with a microorganism. The method comprises culturing the microorganism in the presence of a plurality of glass microparticles to adhere the microorganism to the plurality of glass microparticles, lyophilizing the adhered microorganism to form a plurality of coated micromagmatics, storing and/or transporting the plurality of coated micromagmatics to the target environment, and deploying the plurality of coated micromagmatics in the target environment. The glass microparticles have a median diameter from about 1 μm to about 1000 μm, as determined by laser diffraction according to ISO 13320:2020.

In another aspect, a bioaerosol composition is disclosed. The composition comprises a plurality of aerosolized micromagmatic particles. Each micromagmatic particle comprises a lyophilized microorganism supported on a glass microparticle having a median particle size from about 1 μm to about 1000 μm, as determined by laser diffraction according to ISO 13320:2020

### BRIEF DESCRIPTION OF THE FIGURES

A full and enabling disclosure of the present subject matter, including the best mode thereof to one of ordinary skill in the art, is set forth more particularly in the remainder of the specification, including reference to the accompanying figures in which:

FIG. 1 is a graph showing the growth of Bacillus thuringiensis over time when inoculated using cells preserved on glass cullet.

FIG. 2 illustrates a growth curve of a representative bacterial consortium as a function of time.

### DETAILED DESCRIPTION

Reference will now be made in detail to various embodiments of the disclosed subject matter, one or more examples of which are set forth below. Each embodiment is provided by way of explanation of the subject matter, not limitation thereof. In fact, it will be apparent to those skilled in the art that various modifications and variations may be made in the present disclosure without departing from the scope or spirit of the subject matter. For instance, features illustrated or described as part of one embodiment, may be used in another embodiment to yield a still further embodiment.

In general, the present disclosure is directed to a system for deploying microorganisms to a target environment and methods of deploying such systems. The system generally comprises a plurality of coated micromagmatics formed by adhering a microorganism to a glass microparticle. The system can be deployed in a variety of different ways, including by spraying as an aerosol, as a liquid suspension, or by dispersing within a solid matrix.

The variety of deployment methods allows the system to be used for long-term storage, transport, and delivery of one or more microorganisms in a variety of useful applications. For instance, and without limitation, the system can be formed to include single or a consortium of microorganisms for deployment to mitigate disasters (e.g., oil spills), remediate legacy contamination (e.g., acid mine drainage), utility applications (e.g., wastewater treatment), agricultural applications (e.g., soil restoration), etc. Moreover, as the microorganisms are supported by solid substrates, the materials can be easily recovered following use, preventing the release of the microorganisms retained thereon, as well as preventing overuse of the microorganisms at a deployment site.

The system is environmentally friendly and the glass microparticles are inexpensive and capable of supporting the microorganism(s) of choice. The lyophilization process can be easier using the glass microparticles compared to previously known materials, e.g., unsupported microorganism cultures and substrates not conducive to microorganism attachment, growth, and development. Moreover, the lyophilized microorganism retained on the glass microparticles need only be placed in a receptive environment for growth and development to resume, whereas previously known lyophilized cultures often require a lengthy growth period in a laboratory environment following dormancy. The coated micromagmatics can also provide for long shelf life and easy transport without the need for expensive environmental control, as well as facile reclamation of the materials, following use.

In one embodiment, the glass microparticles can include post-consumer or post-industrial recycled glass. For instance, the glass microparticles can be obtained from glass cullet which, if necessary, can be reduced in size in order to be suitable for forming the micromagmatics. Moreover, in some embodiments, the nature of the glass material allows for further reuse. For instance, following deployment and recovery, the glass microparticles can be autoclaved and reused. For instance, in one embodiment, the coted micromagmatics can be deployed to treat wastewater and thereafter collected for reuse.

As utilized herein, the term “glass” generally refers to a non-crystalline silicate material. In some embodiments, a glass of a porous, solid substrate can include silicon dioxide. However, a glass of a solid substrate is not limited to any particular silicate material and can encompass, without limitation, silicate glasses, borate glasses, phosphate glasses, chalcogenide glasses, naturally occurring glasses such as obsidians and basalt, as well as postcombustion residue ashes containing partially glassy phases, as well as any combinations thereof.

A glass-based substrate can include a glass in conjunction with one or more additional materials, e.g., an amorphous material in conjunction with a secondary material, which can be a crystalline or non-crystalline material, including one or more different glasses. By way of example, and without limitation, a glass-based substrate can include one or more of alumina, alumina hydrate, aplite, feldspar, nepheline syenite, calumite, kyanite, kaolin, cryolite, antimony oxide, arsenious oxide, barium carbonate, barium oxide, barium sulfate, boric acid, borax, anhydrous borax, quicklime, calcium hydrate, calcium carbonate, dolomitic lime, dolomite, finishing lime, litharge, minium, calcium phosphate, bone ash, iron oxide, caustic potash, saltpeter, potassium carbonate, hydrated potassium carbonate, sand, diatomite, soda ash, sodium nitrate, sodium sulphate, sodium silica-fluoride, pyrolysis ash, zinc oxide, or any combination thereof.

In one embodiment, the glass microparticle can include a foamed glass ceramic (FGC), which is commonly described as a synthetic, pumice-like material. FGCs are porous glass and ceramic materials that can be formed almost entirely from waste glass or waste incinerator ashes. An FGC can have increased surface area compared to typical soda-lime glass and can be produced at temperatures significantly lower than the liquidus temperature of the original recycled glass cullet, as well as of a broad range of species, reactivity, and porosity characteristics. Beneficially, FGCs can be produced with less environmental impact than traditional recycled products that require a full “re-melt” of the original glass.

The composition of an FGC can be controlled during pre-firing batching, and the physical properties of the materials can be controlled via chemical composition of the batch in addition to process parameters as described further in U.S. Patent Application Publications 2022/0073416, 2022/0081349, and 2022/0089476 all to Hust et al., which are incorporated herein by reference in their entirety. In some embodiments, an FGC can be formed to include one or more reactive agents that can interact with one or more substances when those substances contact the reactive agents, such as cementitious materials, pozzolanic materials, activated carbon, and/or clayey or zeolitic minerals.

In other embodiments, the glass microparticles are formed from finely powdered glass cullet. For example, the glass microparticles may be obtained by reducing the size of recycled post-consumer or post-industrial glass cullet by, without limitation, utilizing one or more of a hammer mill, jaw crusher, rotary crusher, centrifugal mill, disc mill, ball mill, jet mill, impact mill, high-speed rotary mill, or the like. In some embodiments, an FGC may be subjected to any such equipment in order to reduce the particle size sufficiently.

The median (D50) particle size of the glass microparticles is generally from about 1 μm to about 1000 μm, which allows the particles to serve as a support for microbes while also being small enough to be aerosolized. In some embodiments, the median particle size of the glass microparticles is about 5 μm or greater, in some embodiments about 10 μm or greater, in some embodiments about 50 μm or greater, in some embodiments about 100 μm or greater, and in some embodiments, about 200 μm or greater. In some embodiments, the median particle size of the glass microparticles is about 900 μm or less, in some embodiments about 800 μm or less, in some embodiments about 700 μm or less, in some embodiments about 600 μm or less, and in some embodiments, about 500 μm or less. The median particle size can be measured on a volume basis by laser diffraction according to ISO 13320:2020.

In some embodiments, the coated glass microparticles can be designed to be buoyant in a liquid system. Alternatively, through modification of chemical composition, the glass microparticles can be designed for non-buoyancy, e.g., to fall to the bottom of a liquid target environment, to be held between immiscible liquid phases, to disperse throughout a liquid, etc.

In some embodiments, the glass microparticles may include open-celled porosity, i.e., individual pieces of the media may include passageways extending from an external surface of the piece to the interior and/or to a second external surface of the piece. The porosity can provide high surface area to the microparticles for supporting a high density of microorganism to be developed thereon. The size of the pores can be pre-determined to encourage growth and development of a microorganism of interest. In other embodiments, the glass microparticles do not exhibit open-celled porosity.

In some embodiments, the microparticles can have a single, well-defined porosity. For instance, a silicate aggregate having a single composition with highly homogenous and/or uniform properties, e.g., a single density and a single porosity, can be utilized. In other embodiments, a more complex material can be utilized. For instance, an FGC can include vitreous materials contained at least partially within pores of the microparticles, leading to regions of the particles that are mesoporous (less than about 100 micrometers in cross-section) and/or microporous (less than about 1 micrometer in cross-section).

The bulk density and surface area of the microparticles (e.g., BET surface area) are not particularly limited. For instance, in some embodiments, the microparticles can have a bulk density of from about 0.1 grams per centimeter (g/cc) to about 2 g/cc, such as from about 0.8 to about 4 g/cc, or from about 1 g/cc to about 3 g/cc, in some embodiments. In general, BET surface area of the microparticles can be about 100 square meters per gram (m²/g) or less, in some embodiments about 5 m²/g or less, or about 2 m²/g or less, and in some embodiments, from about 0.1 m²/g to about 1 m²/g.

To form the coated micromagmatics, the glass microparticles are loaded with one or more microorganisms of choice. There is no particular requirement on the types, kinds, or number of microorganisms that can be loaded on the microparticles, and through use of disclosed methods, different lines of bacterial, algal, and fungal cells can be preserved, stored, and regrown after storage for a period of from a few days to several months.

In one embodiment, a microbial consortium can be loaded on a substrate. By way of example, in one embodiment, a substrate material can be inoculated with BioTiger™ (described in U.S. Pat. No. 7,472,747, which is incorporated by reference herein for all purposes), which is a microbial consortia developed by extensive microbiology screening and characterization of samples collected from a waste lagoon. BioTiger™ includes isolates as indicated in Table 1, below, and has been shown to be feasible for degradation of in situ oils, as well as to increase hydrocarbon recovery from oil sands. Several of the bacteria in this strain have also been shown to produce biosurfactants, which can increase the efficiency of the bioremediation process by dispersing the hydrocarbons and causing them to be more easily degraded.

TABLE 1

| Isolate | Identification |
|---|---|
| CZOR-L1B | ALCALIGENES-PIECHAUDII SRS |
| (KN-1) | |
| BP-20 (KN-2) | RALSTONIA PICKETTII SRS |
| CZOR-L1Bsm | PSEUDOMONAS-PUTIDA BIOTYPE B SRS |
| (KN-3) | |
| BPB | FLEXIBACTER CF. SANCTI SRS |
| BPC | PSEUDOMONAS FREDRIKSBERGENSIS SRS |
| BPE | STAPHYLOCOCCUS WARNERI. LMG 19417 SRS |
| BPF | SPHINGOMONAS SRS |
| BPH | SPHINGOMONAS SP. S37 SRS |
| BPI | PHYLOBACTERIUM SRS (α PROTEOBACTERIUM |
| | TA-A1) |
| BPJ | SERRATIA FICARIA SRS (α PROTEOBACTERIUM |
| | TA12-21) |
| BPK | AGROBACTERIUM TUMEFACIENS SRS |
| BPL | RHIZOBIUM SP. SDW045 SRS |

Other microorganisms which may be carried by the microparticles described herein can include, without limitation, Bacillus thuringiensis, Bacillus subtilis, Bacillus cereus, Bradyrhizobium spp., Thiobacillus ferrooxidans, Azospirillum spp., Azotobacter spp., Pseudomonas Putida, Pseudomonas fluorescens, Enterobacter spp., Penicillium spp., Aspergillus spp., Gluconobacter oxydans, Gluconacetobacter xylinus, Nitrosomonas spp., Nitrosococcus spp., Clostridium spp., Acidovorax delafieldii, Rhodococcus spp., Alcanivorax borkumensis, Halomonas spp., Vibrio gazogenes, Marinobacter hydrocarbonoclasticus, and Sphingomonas paucimobilis. Moreover, as with the BioTiger™ consortium mentioned herein, while individual microorganisms can be utilized alone, in some embodiments, a mixture of microorganisms can be utilized, and the resulting product can exhibit enhanced effect. For instance, one or more oil-degrading bacteria as are known in the art can be loaded on the microparticles, optionally in conjunction with additional microorganisms, and the resulting product can exhibit a synergetic effect.

The method utilized for immobilization of the microorganism(s) on the microparticles is not particularly limited and generally includes culturing the microorganisms in a suitable culture environment in the presence of the glass microparticles. In some embodiments, an immobilization process can be similar to that of natural systems in the formation of biofilms. Beneficially, through utilization of disclosed materials and methods, microorganisms that do not preferentially live in a biofilm form can be immobilized on glass microparticles as a biofilm.

Biofilms are ubiquitous throughout the environment, both natural and synthetic. A biofilm is a consortium of sessile microorganisms that have established a three-dimensional community including of a combination of prokaryotic or eukaryotic cells embedded in a microbially produced matrix of extracellular polymeric substances (EPS). The EPS can include proteins, polysaccharides, humic substances, extracellular DNA, as well as additional molecules. Social and physical interactions occur intercellularly in conjunction with the EPS creating a unique and emergent lifestyle, distinctly different from that of a free-living microorganism. The unique properties of biofilms in comparison to their free-living cellular counterparts are well documented and can include increased antibiotic resilience, increased mutations, consortia building, and increased quorum-sensing-regulated mechanisms. Thus, providing a microorganism amendment in the form of a solid supported biofilm can have an advantage in bioremediation applications as it can provide the microorganisms in a high surface area and high-density format that can increase the interaction with environmental contaminants and deployment capacity among other benefits, in addition to providing the ability to incorporate beneficial nutrients for the microorganisms into/onto the microparticles in conjunction with the microorganisms.

The microparticles can also provide a route to modification of bulk characteristics of the amendments during use. For instance, the coated microparticles can be engineered such that they sink to the bottom of a liquid medium after deployment. However, it should also be understood that the buoyancy of the microparticles can be altered such that the microparticles can float on top of the liquid medium.

Prior to lyophilizing, nutrients (nitrogen (N), phosphorous (P), carbon (C), or other key minerals) can be added to the materials for specific applications as an amendment. For example, in an oil-contaminated environment with high carbon content, a preparation with added N and P could be advantageous. Such amendments can be easily added on or with the composite material and can be application (e.g., site) specific.

Following inoculation, the supported microorganisms can be lyophilized. In general, the lyophilization process can encompass a standard lyophilization process, e.g., with treatment of 10% glycerin, flash-freezing below the eutectic point of the sample (e.g., at −80° C.), and freeze drying. However, there are microorganisms that may benefit from a more modified process, e.g., a rapid lyophilization method or preservation using dimethyl sulfoxide, as is generally known in the art.

The micromagmatics including the lyophilized microorganisms thereon can be stored as desired, e.g., up to several months or more. Beneficially, the storage environment does not require any specialized environmental conditions. For instance, the micromagmatics can be stored in air at standard temperature and pressure conditions.

The micromagmatics can likewise be shipped and deployed without the need for any specialized environmental conditions. For instance, the micromagmatics need not be subjected to laboratory processing to revitalize the preserved microorganisms. In many embodiments, the micromagmatics can simply be located in the deployment area, following which the retained microorganisms can begin to grow and develop while retained on the glass surface.

The coated micromagmatics can be deployed by various methods, for example, by spraying (e.g., as a bioaerosol), as a suspension in a liquid (e.g., water), or in a solid matrix material (e.g., soil), allowing them to be employed in a wide variety of applications. For example, the coated microparticles are useful for land, surface and subsurface water, and aerosol applications. Advantageously, the present inventors discovered that the material is resistant to environmental degradation, allowing for such versatility, especially when used in marine or subsurface applications.

In one embodiment, the system is deployed as a bioaerosol comprising a gaseous suspension of the plurality of micromagmatics. As used herein, the bioaerosol does not necessarily need to form a true suspension in the gaseous medium and can also be a plurality of fine particles propelled by a gaseous stream. For example, the system can be deployed by connecting a reservoir of the coated micromagmatic to a stream of air (e.g., from a blower or air gun) to “aerosolize” the particles and spray them onto a target environment.

In some embodiments, the coated microparticles can be dispersed within a liquid (e.g., water) to form a suspension. The suspension can optionally include additional components that may be useful for a desired application or may act synergistically with the microorganisms supported on the microparticles. For example, any suitable additional inert ingredient that is used as a carrier, solvent, diluent, emulsifier, dispersant, stabilizer, suspending agent, penetrant, or the like can also be included in the liquid composition. The liquid composition can be deployed by spraying or dumping it on a target environment or, if the target environment is another liquid, mixing the suspension with the target liquid. The liquid composition can also be deployed as an aerosol. For example, the liquid composition can be mixed with a propellant in a pressurized container and sprayed through an aerosol nozzle as is known in the art.

In another embodiment, the coated micromagmatics are dispersed in a solid matrix. For example, the particles can be mixed into a soil composition which can be used for agricultural purposes. In one embodiment, for instance, the micromagmatics can include Bacillus thuringiensis, a natural insecticide, and can be mixed in soil. Other useful components (e.g., plant nutrients, fertilizers, perlite, etc.) can be included in the composition as well. Alternatively, in some embodiments, the micromagmatics may be dispersed in a biodegradable polymeric matrix.

As explained above, there are many potential target environments and applications for deploying the system of coated micromagmatics. Some examples of applications include those in the oil, gas, and mining industries, agricultural applications, wastewater treatment systems, defense, biotechnology, and bioremediation projects where effective delivery of active microbial species is critical.

In one embodiment, for example, Bacillus thuringiensis is supported on the glass microparticles for use in commercial, environmental, and agricultural pest control applications. As mentioned above, this method of deployment can be coordinated with planting season to mix in soil with water and other key nutrients to facilitate plant growth.

In another embodiment, BioTiger™ bacterial consortium is supported on the glass microparticles for biodegradation of hydrocarbons (e.g., for remediation of oil spills). One advantage of the system is that it can be deployed in remote areas (e.g., by deploying from an aircraft) and can be formulated to float on aqueous environments, which is particularly useful to help remediate oil spills. Such materials can also be used for bioremediation of contaminated soils where bioavailability is an issue.

In another embodiment, the coated microparticles can be used in wastewater treatment applications. Many wastewater treatment plants make use of activated sludges, aerated lagoons, ponds, wetlands, and biological reactions among other techniques. However, the coated microparticles described herein provide a means to add a biological component to the wastewater stream, via a solid delivery method of lyophilized bacteria that can be easily recovered, as the microparticles can be designed to float to the top of a reservoir or sink to the bottom of a tank.

In some embodiments, the microparticles can be engineered for subsurface applications by employing anaerobic bacteria.

In another embodiment, the particles can be applied by spraying at a mining site over areas of environmental concern, allowing workers to have minimal contact with potentially hazardous sites.

Application via spraying or aerosolization is particularly useful as the particles can be distributed over large areas and in remote or inconvenient locations such as difficult terrain. For example, a bioaerosol containing the coated micromagmatics can be efficiently sprayed over a large area by spraying from an aircraft.

The microparticles can advantageously be engineered for specific applications. For example, recycled glass could be used in the manufacture resulting in particles with increased weight and density, so they settle or mix better in soils, sediments, or liquid matrices. In addition, microparticles could be manufactured with light weight biodegradable polymers, so they float on surfaces where they can interact with oil spills. In some embodiments, the micromagmatics can be modified with other components, such as iron, fertilizers, organic carbon, etc., for specific targets. The microparticles can also be applied to waste sites such as landfills to increase biodegradation of organics to increase subsidence as well as odor control. Micromagmatics can be varied in size and density so they can be applied in aqueous agricultural spray irrigation systems or from sprayers mounted on helicopters, drones, or planes.

The present invention may be better understood with reference to the examples set forth below.

### Example 1

BioTiger™ bacterial consortium, Bacillus thuringiensis (ATCC 35646) and Escherichia coli K-12 (ATCC 25404), were routinely cultured on Reasoner's 2A (R2A; Fisher Scientific) media containing the following per liter of water: 0.5 g casein acid hydrolysate, 0.5 g dextrose, 0.3 g K₂HPO₄, 0.024 g MgSO₄, 0.5 g proteose peptone, 0.3 g sodium pyruvate, 0.5 g soluble starch, and 0.5 g yeast extract. The media was buffered to a pH of 7.2±0.2 at 25° C. Routine bacteria stocks were cultured at room temperature on a shaker plate at 100 rpm. All additional experiments using bacteria were conducted using R2A media or agar prepared with 15 g/L agar (Fisher Scientific), as necessary. To test for compatibility with different cell types, an environmental algal sample was used. The algal sample was maintained using Bushnell Haas broth containing the following per liter of water: 0.2 g MgSO₄, 0.02 g CaCl₂, 1.0 g KH₂PO₄, 1.0 g K₂HPO₄, 1.0 g NH₄NO₃, and 0.05 g FeCl₃ adjusted to a final pH of 7+0.2.

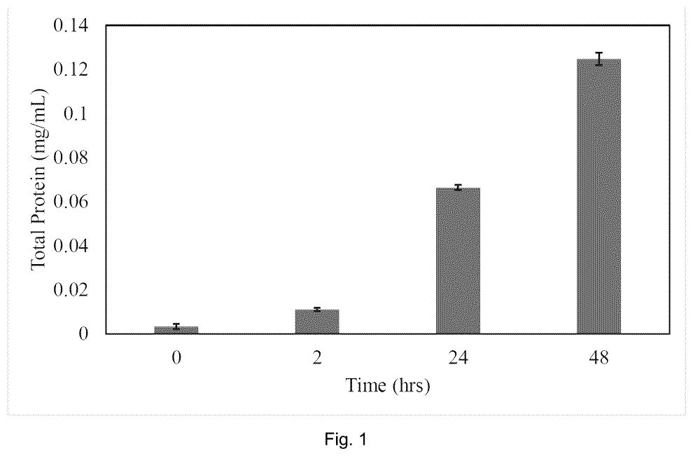

To determine the effectiveness of the glass microparticles as a substrate for biofilm growth, 3 g of sterile glass particles were placed into 100 mL of sterile R2A media. This media was then inoculated with 1 mL of log phase cells (either BioTiger™, Bacillus thuringiensis, Escherichia coli K-12, or the algae). The samples were incubated at room temperature on a rotary shaker plate at 100 rpm for one week. After samples had become laden with cell mass, a sterile solution of 80% glycerol was added, bringing the overall concentration of glycerol to 20% following which the samples were allowed to rest for five minutes. The coated micromagmatics were then removed from the solution and placed in a −80° C. freezer. After freezing, the samples were lyophilized in a Labconco™ lyophilizer and freeze-dried over a 48-hour period. Samples were then stored in a refrigerator at 4° C. FIG. 1 shows the results of a total protein assay of Bacillus thuringiensis over time when inoculated using cells preserved on glass cullet. As shown, the bacteria replicated efficiently on the glass cullet. Preservation success was evaluated when samples were examined using a Zeiss stereoscope.

To determine the success of storage, 8-10 of the coated micromagmatics were placed into R2A media 1, 3, 7, 14, 21, 28, 56, and 84 days after lyophilization. Growth was indicated by an increase in turbidity (OD600) over the course of 72 hours at room temperature and shaking at 100 RPM.

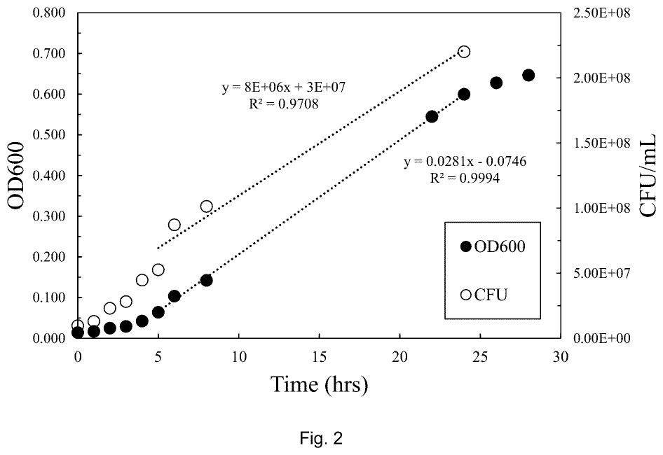

To determine the rate of regrowth of preserved micromagmatics, a series of micromagmatics were incubated at 0, 2, 6, 24, 48, 72, and 196 hours with BioTiger™ prior to the preservation process, and then preserved as described above. Micromagmatics preserved at each time point were placed in 75 mL of sterile R2A broth and the optical density (OD600) was measured over time using a visible spectrophotometer. Previously, the OD600 of BioTiger™ was correlated to colony forming units (CFUs) in triplicate, on R2A agar. The resulting growth curves are illustrated in FIG. 2. As indicated, trend lines calculated from 5-24 hours show a linear growth with an R2 of 0.9994 and 0.9708 for the optical density and CFU samples, respectively. Error bars were within the size of the symbols shown.

While certain embodiments of the disclosed subject matter have been described using specific terms, such description is for illustrative purposes only, and it is to be understood that changes and variations may be made without departing from the spirit or scope of the subject matter.

1. A system for deploying a microorganism to a target environment, the system comprising the bioaerosol composition of claim 20.

2. The bioaerosol composition of claim 20, wherein the glass microparticle comprises recycled glass cullet.

3. The bioaerosol composition of claim 20, wherein the lyophilized microorganism is a component of a lyophilized biofilm.

4. The bioaerosol composition of claim 20, wherein the microorganism comprises a bacterium.

5. The bioaerosol composition of claim 4, wherein the bacterium comprises Bacillus thuringiensis or an oil degrading bacterium.

6. The system of claim 1, wherein the plurality of micromagmatic particles are suspended in a liquid.

7. The system of claim 1, wherein the plurality of micromagmatic particles are dispersed in a soil matrix.

8. The system of claim 1, wherein the plurality of micromagmatic particles are aerosolized.

9. A method for treating a target environment with a microorganism, the method comprising:
culturing the microorganism in the presence of a plurality of glass microparticles having a median diameter from about 1 μm to about 1000 μm as determined by laser diffraction according to ISO 13320:2020, wherein upon the culturing, the microorganism is adhered to the plurality of glass microparticles;
lyophilizing the adhered microorganism to form a plurality of coated micromagmatics;
aerosolizing the plurality of coated micromagmatics to form the bioaerosol composition of claim 20; and
storing and/or transporting the plurality of coated micromagmaties bioaerosol composition to the target environment.

10. The method of claim 9, further comprising deploying the bioaerosol composition in the target environment.

11. The method of claim 10, wherein following the deployment, the lyophilized microorganism grows and develops.

12. The method of claim 10, wherein deploying the bioaerosol composition comprises spraying the bioaerosol composition on or within the target environment.

13. The method of claim 10, wherein deploying the coated micromagmatics comprises mixing the plurality of coated micromagmatics with a liquid to form a suspension and contacting the target environment with the suspension.

14. The method of claim 10, wherein deploying the coated micromagmatics comprises forming a composition comprising the plurality of coated micromagmatics and soil and contacting the target environment with the composition.

15. The method of claim 9, wherein the target environment comprises a contaminated soil.

16. The method of claim 9, wherein the target environment comprises a contaminated water.

17. The method of claim 9, wherein the target environment comprises an agricultural soil.

18. The method of claim 9, further comprising reducing the size of recycled glass cullet to form the glass microparticles.

19. The method of claim 10, further comprising collecting the plurality of micromagmatics after deploying them.

20. A bioaerosol composition comprising a plurality of aerosolized micromagmatic particles, each micromagmatic particle comprising a lyophilized microorganism supported on a glass microparticle having a median particle size from about 1 μm to about 1000 μm as determined by laser diffraction according to ISO 13320:2020.
